(19) United States
(12) Patent Application Publication
(10) Pub. No.: US 2024/0067682 A1
(43) Pub. Date: Feb. 29, 2024

(54) PEPTIDE HAVING PLANT STEM CELL-INDUCING EFFECT AND PLANT PEST RESISTANCE-INDUCING EFFECT

(71) Applicants: KYOTO PREFECTURAL PUBLIC UNIVERSITY CORPORATION, Kyoto-shi, Kyoto (JP); KYOTO SANGYO UNIVERSITY, Kyoto-shi, Kyoto (JP)

(72) Inventors: Masahiko SATO, Kyoto-shi, Kyoto (JP); Issei OHSHIMA, Kyoto-shi, Kyoto (JP); Tomoko HIRANO, Kyoto-shi, Kyoto (JP); Seisuke KIMURA, Kyoto-shi, Kyoto (JP)

(21) Appl. No.: 18/025,541

(22) PCT Filed: Sep. 16, 2021
(86) PCT No.: PCT/JP2021/034176

§ 371 Date: Mar. 9, 2023

(30) Foreign Application Priority Data
Sep. 17, 2020 (JP) 2020-156361

(51) Int. Cl. C07K 14/435; C12N 15/82

(52) U.S. Cl. CPC C07K 14/43563; C12N 15/8262

(57) ABSTRACT

Disclosed are a partial peptide of cysteine-rich secretory protein, antigen 5, and pathogenesis-related 1 (CAP), consisting of an amino acid sequence of the conserved region of CAP, the peptide having at least one effect selected from the following effects: a plant stem cell-inducing effect, a plant environmental stress tolerance-enhancing effect, a plant pest resistance-inducing effect, a germination-promoting effect, a plant transformation efficiency-enhancing effect, and a plant growth-promoting effect; and a plant stem cell inducer, an environmental stress enhancer, a plant pest resistance inducer, a germination promoter, a transformation efficiency enhancer, and a plant growth promoter, all of which comprise the peptide.

### TECHNICAL FIELD

The present invention relates to a peptide, a plant stem cell inducer, an environmental stress enhancer, a plant pest resistance inducer, a germination promoter, a transformation efficiency enhancer, and a plant growth promoter. The present invention also relates to a method for producing plant stem cells, a method for enhancing the environmental stress tolerance of a plant body, a plant pest control method, a method for promoting plant seed germination, a method for transforming a plant, and a method for promoting the growth of a plant body.

### BACKGROUND OF THE INVENTION

Methods to promote stem cell transformation in plants include methods using plant hormones. A mass of undifferentiated cells produced by such a method is called a “callus.” However, only a few plants can increase such callus tissues, and there is a severe problem that it takes several weeks to generate callus tissues.

In the previous studies in angiosperms, such as Arabidopsis thaliana, it has been possible to produce meristematic tissues containing stem cells by creating genetically modified plants by transformation of specific genes, such as the KNOX family. However, since only some of differentiated cells that repeat cell division can be transformed into stem cells, it is difficult to identify cells having an ability to become stem cells, and the mechanism that transforms differentiated cells into stem cells is not well understood. In addition, this approach cannot be applied to plants that are difficult to transform, and there is a problem that it is difficult to use genetically modified plants obtained by transformation outside the laboratory.

It has been possible to generate freezing-tolerant and drought-tolerant plants, and pest-resistant plants by transforming and controlling specific genes, such as transcription factors related to freezing tolerance and drought tolerance, and transcription factors related to disease resistance. However, such an approach cannot be applied to plants that are difficult to transform, and there is also a problem that it is difficult to use genetically modified plants obtained by transformation outside the laboratory.

There are also conventional methods to protect plants from pests by using pesticides. However, the use of pesticides has a concern of disadvantages, such as health effects, negative environmental impact, and increasing pests harboring pesticide resistance.

It has been possible to promote germination by treating seeds with a plant hormone, gibberellin. However, in general, the germination rate of plant seeds harvested more than 3 years before is significantly reduced, and almost no germination occurs after 5 years or more.

In addition, it has been possible to improve transformation efficiency by introducing a plasmid that expresses GABA aminotransferase gene, which has a function to suppress GABA generation, into Agrobacterium. However, even when a plasmid that expresses GABA aminotransferase gene is used, the efficiency is increased up to only about 3-fold.

PTL 1 and PTL 2 have reported other methods to promote stem cell transformation in plants.

PTL 1 discloses a method for producing dedifferentiation cells, comprising a dedifferentiation induction step of subjecting all or a part of a plant body to heat shock treatment at 28° C. to 42° C. for 30 minutes to 5 days, and a culturing step of culturing the plant body after the dedifferentiation induction step at an ordinary growth temperature for the plant.

Further, PTL 2 discloses a method for producing a plant body, comprising producing, in a plant body, a chimeric protein that fuses a transcription factor promoting the transcription of a gene for inhibiting dedifferentiation or redifferentiation with a functional peptide for transforming any transcription factor to a transcription-inhibiting factor, thereby promoting the dedifferentiation or redifferentiation of the plant body.

### CITATION LIST

### Patent Literature

PTL 1: JP2015-208256A
PTL 2: JP2012-187040A

### SUMMARY OF INVENTION

### Technical Problems to be Solved by the Present Invention

An object of the present invention is to provide a peptide having a plant stem cell-inducing effect, a plant environmental stress tolerance-enhancing effect, a plant pest resistance-inducing effect, a germination-promoting effect, a plant transformation efficiency-enhancing effect, and a plant growth-promoting effect, as well as a plant stem cell inducer, an environmental stress enhancer, a plant pest resistance inducer, a germination promoter, a transformation efficiency enhancer, and a plant growth promoter, all of which comprise the peptide. Another object of the present invention is to provide a method for producing plant stem cells, a method for enhancing the environmental stress tolerance of a plant body, a plant pest control method, a method for promoting plant seed germination, a method for transforming a plant body, and a method for promoting the growth of a plant body, all of which use the above peptide.

### Solution to Solve the Problems

As a result of intensive studies to achieve the above objects, the present inventors found that differentiated cells can be transformed into stem cells only by immersing plants in a solution containing a synthetic peptide consisting of the conserved region of cysteine-rich secretory protein, antigen 5, and pathogenesis-related 1 (CAP) for a short period of time. The inventors also found that plants with enhanced freezing tolerance, drought tolerance, and pest resistance can be produced by immersing plants in a solution containing the peptide, or by applying a solution containing the peptide to plants. In addition, the inventors found that germination can be promoted by immersing plant seeds in a solution containing the peptide, transformation efficiency can be enhanced by performing plant transformation in the presence of the peptide, and the growth of plants can be promoted by applying a solution containing the peptide to the plants.

The present invention has been completed by conducting further studies based on these findings. The present invention provides the following peptide, plant stem cell inducer, environmental stress enhancer, plant pest resistance inducer, method for producing plant stem cells, method for enhancing the environmental stress tolerance of a plant body, plant pest control method, and the like.

Item 1. A peptide that is a partial peptide of cysteine-rich secretory protein, antigen 5, and pathogenesis-related 1 (CAP), consisting of an amino acid sequence of the conserved region of CAP, the peptide having at least one effect selected from the following effects: a plant stem cell-inducing effect, a plant environmental stress tolerance-enhancing effect, a plant pest resistance-inducing effect, a germination-promoting effect, a plant transformation efficiency-enhancing effect, and a plant growth-promoting effect.

Item 2. The peptide according to Item 1, wherein CAP is secreted from a gall-inducing insect.

Item 3. A peptide comprising an amino acid sequence represented by formula (I):

(X¹)ₙTQX²X³W (I)
wherein X¹ represents phenylalanine or tyrosine, X² represents isoleucine or valine, X³ represents isoleucine or valine, and n represents 0 or 1;
and having 25 or less amino acid residues.

Item 4. The peptide according to any one of Items 1 to 3, which is a peptide consisting of all or a part of an amino acid sequence represented by SEQ ID NO: 1.

Item 5. A plant stem cell inducer comprising the peptide according to any one of Items 1 to 4.

Item 6. A plant environmental stress tolerance enhancer comprising the peptide according to any one of Items 1 to 4.

Item 7. The environmental stress tolerance enhancer according to Item 6, wherein the environmental stress tolerance is drought tolerance, osmotic tolerance, salt tolerance, or freezing tolerance.

Item 8. A plant pest resistance inducer comprising the peptide according to any one of Items 1 to 4.

Item 9. A germination promoter comprising the peptide according to any one of Items 1 to 4.

Item 10. A plant transformation efficiency enhancer comprising the peptide according to any one of Items 1 to 4.

Item 11. A plant growth promoter comprising the peptide according to any one of Items 1 to 4.

Item 12. A method for producing plant stem cells, comprising applying the peptide according to any one of Items 1 to 4 to a plant body.

Item 13. A method for enhancing the environmental stress tolerance of a plant body, comprising applying the peptide according to any one of Items 1 to 4 to the plant body.

Item 14. A plant pest control method comprising applying the peptide according to any one of Items 1 to 4 to a plant body.

Item 15. A method for promoting plant seed germination, comprising applying the peptide according to any one of Items 1 to 4 to plant seeds.

Item 16. A method for transforming a plant body, comprising transforming the plant body in the presence of the peptide according to any one of Items 1 to 4.

Item 17. A method for promoting the growth of a plant body, comprising applying the peptide according to any one of Items 1 to 4 to the plant body.

### Advantageous Effects of the Invention

Since the peptide of the present invention has an excellent plant stem cell-inducing effect, plant environmental stress tolerance (e.g., drought tolerance, osmotic tolerance, or freezing tolerance)-enhancing effect, plant pest resistance-inducing effect, germination-promoting effect, plant transformation efficiency-enhancing effect, and plant growth-promoting effect, exposing plants to the peptide of the present invention can transform differentiated cells of the plants into stem cells, enhance the environmental stress tolerance (e.g., drought tolerance, osmotic tolerance, or freezing tolerance) of the plants, enhance the pest resistance of the plants, promote the germination of plant seeds, enhance plant transformation efficiency, and promote the growth of the plant bodies.

Since most species of animals and plants have the CAP gene in common, most plants also have receptors for CAP. Therefore, the peptide of the present invention is expected to be able to, for various plants, induce stem cell transformation, enhance environmental stress tolerance, enhance pest resistance, promote germination, enhance transformation efficiency, and promote growth.

Stem cell transformation requires only short-term exposure of plants to the peptide of the present invention, and is extremely simple and fast-acting. Further, since all cells infiltrated with the peptide of the present invention are transformed into stem cells, it is easy to identify cells that have differentiated into stem cells.

The peptide of the present invention does not require the process of transformation, and thus can basically be applied to any plants, and can be used both inside and outside the laboratory. Further, the peptide of the present invention is not toxic to living organisms unlike pesticides, and is decomposed by microorganisms in the environment, which causes no problem with residue.

The peptide of the present invention makes it possible to germinate seeds harvested more than 8 years before or not germinated by gibberellin treatment. Further, the peptide of the present invention not only significantly enhances transformation efficiency, but also allows transformation of plants that have not been able to transform thus far. In addition, the peptide of the present invention has an effect of promoting the growth of plants, which makes it possible to economically increase the production of crops without excessive fertilization.

### BRIEF DESCRIPTION OF DRAWINGS

FIG. 1 shows the alignment of the CAP protein sequences of Arabidopsis thaliana (At), Schlechtendalia chinensis (Sc), Borboryctis euryae (Be), Caloptilia cecidophora (Cc), and Caloptilia ryukyuensis (Cr).

FIG. 2 shows the results obtained in Test Example 1. Arabidopsis thaliana expressing stem cell marker PLT1-GFP was immersed in water (A, C) or 600 nM of the peptide aqueous solution (B, D) for 2 days. (A, B) are GFP fluorescent images, and (C, D) are bright field images.

FIG. 3 shows the effect of imparting osmotic tolerance and drought tolerance by treatment with the peptide of the present invention. The upper figure shows the growth on an excess mannitol-containing medium after immersion in 600 nM CAP peptide for 5 minutes. The values in the graphs are mean values 9.50 (S.D.=1.61) and 11.69 (S.D.=1.55), and *p<0.05 (Student's t-test). The lower figure shows the growth on an excess sorbitol-containing medium after immersion in 600 nM CAP peptide overnight. The values in the graphs are mean values 5.17 (S.D.=1.73) and 7.71 (S.D.=1.39), and *p<0.05 (Student's t-test).

FIG. 4 shows the effect of inhibiting the growth of plant pathogen Pseudomonas syringae pv tomato DC3000 (DC3000) by spraying Arabidopsis thaliana leaves with water or a CAP peptide solution prior to the bacterial infection. Two days after infection, DC3000 obtained from leaf fragments (cut into 4-mm-diameter circles) was diluted, and then the diluted solutions were spotted on LB medium containing kanamycin, and incubated at 28° C. for 24 hours (A). Then, the number of bacteria per unit area was counted (B).

FIG. 5 shows the effect of inhibiting the growth of plant pathogen Pseudomonas syringae pv tomato DC3000 (DC3000) by spraying the peptide to Arabidopsis thaliana leaves with water (Control) or a 60 nM CAP peptide solution in advance. The photographs of both cases were taken at 0, 4, and 13 days after infection of water-treated or CAP peptide-treated Arabidopsis thaliana with plant pathogen DC3000.

FIG. 6 shows the seeds collected from one individual of Arabidopsis thaliana of FIG. 5, showing the increase in the seed yield due to CAP peptide treatment.

FIG. 7 shows photographs showing the effect of CAP peptide on infection of Brassica rapa with plant pathogen Pseudomonas syringae DC3000 (DC3000). Brassica rapa leaves were sprayed with water (Control) or a 60 nM CAP peptide solution in advance, and one day later, inoculated with plant pathogen DC3000. The photographs of both cases were taken at 0, 8, and 13 days after infection.

FIG. 8 shows photographs showing the effect of CAP peptide to inhibit the growth of the pathogen in Brassica rapa. Brassica rapa leaves were sprayed with water or a CAP peptide solution in advance, and one day later, inoculated with plant pathogen Pseudomonas syringae pv tomato DC3000 (DC3000). 2 days after infection, DC3000 obtained from leaf fragments cut into 4-mm-diameter circles was diluted, spotted on LB medium containing kanamycin, and incubated at 28° C. for 24 hours.

FIG. 9 shows the CAP consensus sequence of Schlechtendalia chinensis, Caloptilia cecidophora, Borboryctis euryae, Caloptilia ryukyuensis, Rhus javanica, and Arabidopsis thaliana.

FIG. 10 is a graph showing the effect of CAP6 peptide on the infection with plant pathogen Pseudomonas syringae DC3000.

FIG. 11 is a graph showing the effect of promoting germination by treating Arabidopsis thaliana seeds with CAP peptide. The horizontal axis indicates the concentration of CAP peptide. n=126 (0 μM CAP, 0 mg/L GA), 143 (1 μM CAP, 0 mg/L GA), 107 (10 μM CAP, 0 mg/L GA), 53 (100 μM CAP, 0 mg/L GA), 115 (1 μM CAP, 0.1 mg/L GA), 74 (1 μM CAP, 0.1 mg/L GA), 68 (10 μM CAP, 0.1 mg/L GA), 58 (100 μM CAP, 0.1 mg/L GA)

FIG. 12 is a graph showing the number of regenerated plantlets obtained by transforming hard-to-transform plant Rorippa aquatica in the presence of CAP peptide. The horizontal axis indicates the concentration of CAP peptide. The value in the graph indicates the percentage of transformed individuals. n=15

FIG. 13 is a graph showing the effect of promoting growth by treating cherry tomato Sun Cherry seedlings with CAP peptide. The values in the graph are mean±SD, and n=4 (control), 5 (CAP peptide treatment).

### DESCRIPTION OF EMBODIMENTS

Embodiments of the present invention are described below.

In the present specification, the term “comprise” includes the meanings of “essentially consisting of” and “consisting of.”

In the present specification, the gene encoding CAP is referred to as “CAP gene,” and the simple expression “CAP” means the protein. In the present specification, amino acids are expressed in one-letter notation, unless otherwise specified.

The peptide of the present invention, in an embodiment, is a partial peptide of cysteine-rich secretory protein, antigen 5, and pathogenesis-related 1 (CAP), consisting of an amino acid sequence of the conserved region of CAP, and having at least one of a plant stem cell-inducing effect, a plant environmental stress tolerance-enhancing effect, a plant pest resistance-inducing effect, a germination-promoting effect, a plant transformation efficiency-enhancing effect, and a plant growth-promoting effect (hereinafter also referred to as “peptide 1 of the present invention”).

CAP (cysteine-rich secretory protein, antigen 5, and pathogenesis-related 1) belongs to the CAP superfamily. Most species of animals and plants have CAP in common, and some species of animals and plants may have many CAP proteins. Amino acid sequences of CAP are registered on the NCBI website as, for example, Ref Seq Accession No. NP_197985 (SEQ ID NO: 2), NP_175427 (SEQ ID NO: 3), NP_195893 (SEQ ID NO: 4), NP_171638 (SEQ ID NO: 5), NP_680450 (SEQ ID NO: 6), NP_194309 (SEQ ID NO: 7), NP_194308 (SEQ ID NO: 8), NP_187570 (SEQ ID NO: 9), NP_194875 (SEQ ID NO: 10), NP_201460 (SEQ ID NO: 11), NP_194761 (SEQ ID NO: 12), NP_175428 (SEQ ID NO: 13), NP_188603 (SEQ ID NO: 14), NP 195098 (SEQ ID NO: 15), NP 195097 (SEQ ID NO: 16), NP 192524 (SEQ ID NO: 17), NP_179587 (SEQ ID NO: 18), NP_195099 (SEQ ID NO: 19), and NP_179588 (SEQ ID NO: 20) (all Arabidopsis thaliana).

Further, the unknown amino acid sequences of CAP other than those registered on the database as described above can be obtained by known methods. As such a method, for example, RNA is extracted from organisms, RNA-Seq analysis is performed, and transcripts annotated as CAP are obtained as the amino acid sequences of CAP peptides.

CAP of the present invention widely includes CAP having a naturally occurring amino acid sequence, and variants obtained by substitution, addition, deletion, and/or insertion of one or two or more amino acids in the amino acid sequence of this CAP, and having biological activities equivalent to those of proteins consisting of the above amino acid sequence.

The range of “one or two or more” is not particularly limited, and means, for example, 1 to 50, preferably 1 to 25, more preferably 1 to 12, even more preferably 1 to 9, and particularly preferably 1 to 5. Techniques of substitution, addition, deletion, and/or insertion of one or two or more amino acids in specific amino acid sequences are known.

Peptide 1 of the present invention is a part of the amino acid sequence of CAP, and consists of an amino acid sequence of the conserved region. The conserved region of CAP is generally located near the C-terminal and can be determined by multiple alignment of CAP derived from various organisms.

Multiple alignment can be performed using analysis tools available on the market or via telecommunication lines (Internet). The number of amino acid residues in the conserved region of CAP is not particularly limited, and is, for example, 6 to 10, 6 to 11, 6 to 12, 6 to 13, 6 to 14, 6 to 15, 6 to 16, 6 to 17, 6 to 18, 6 to 19, 6 to 20, 6 to 21, 6 to 22, 6 to 23, 6 to 24, or 6 to 25.

CAP may be derived from any of microorganisms, animals, and plants. Among these, CAP produced by insects is preferred, and CAP secreted by gall-inducing insects is more preferred. Examples of gall-inducing insects include, but are not particularly limited to, Schlechtendalia chinensis, Borboryctis euryae, Caloptilia cecidophora, Caloptilia ryukyuensis, and the like.

In the present invention, the “plant stem cell-inducing effect” refers to the effect of transforming plant differentiated cells into stem cells (dedifferentiation) or the effect of maintaining stem cells (undifferentiated cells) without differentiation. Plant stem cells refer to plant cells having pluripotency and cell division ability, and include a callus.

In the present invention, the “plant environmental stress tolerance-enhancing effect” refers to the effect of rendering plants substantially immune to undesired effects of environmental stress or the effect of reducing the effects. The environmental stress as mentioned herein is not particularly limited, and examples include salt stress, drought stress, osmotic stress, temperature (e.g., high temperature, low temperature, or freezing) stress, and the like. The plant environmental stress tolerances in the present invention are preferably drought tolerance, osmotic tolerance, salt tolerance, and freezing tolerance.

In the present invention, the “plant pest resistance-inducing effect” refers to the effect of inducing the resistance of plants to pathogens and pests. Induction of pest resistance also includes enhancement of pest resistance.

In the present invention, the “transformation efficiency-enhancing effect” refers to the effect of enhancing the ratio of transformed plants, and also means that plants that have been unable, or almost unable, to transform can be transformed. The method of transformation is preferably an Agrobacterium method.

In the present invention, the “plant growth-promoting effect” includes expansion and hypertrophy of stems, expansion and hypertrophy of roots, growth of leaves, promotion of branching, increase in the fruiting rate, growth of fruit, and the like.

Peptide 1 of the present invention has at least one of a plant stem cell-inducing effect, a plant environmental stress tolerance-enhancing effect, a plant pest resistance-inducing effect, a germination-promoting effect, a plant transformation efficiency-enhancing effect, and a plant growth-promoting effect; preferably at least 2, 3, 4 or 5 of them; and more preferably all of them.

The peptide of the present invention, in another embodiment, comprises an amino acid sequence represented by formula (I):

(X′)ₙTQX²X³W (I)
wherein X¹ represents phenylalanine or tyrosine, X² represents isoleucine or valine, X³ represents isoleucine or valine, and n represents 0 or 1;
and has 25 or less amino acid residues (hereinafter also referred to as “peptide 2 of the present invention”).

In peptide 2 of the present invention, n is preferably 1, and X¹ is preferably phenylalanine. As shown in the Examples provided later, in CAP secreted by gall-inducing insects, X¹ is phenylalanine. Further, X² is preferably isoleucine, and X³ is preferably valine.

The number of amino acid residues in peptide 2 of the present invention is, for example, 25 or less, 24 or less, 23 or less, 22 or less, 21 or less, 20 or less, 19 or less, 18 or less, 17 or less, 16 or less, 15 or less, 14 or less, 13 or less, 12 or less, 11 or less, or 10 or less.

In peptide 2 of the present invention, the part other than the amino acid sequence represented by formula (I) is composed of any amino acids. Any amino acids used herein are not particularly limited as long as the effects of the present invention (plant stem cell-inducing effect, plant environmental stress tolerance-enhancing effect, plant pest resistance-inducing effect, germination-promoting effect, plant transformation efficiency-enhancing effect, and plant growth-promoting effect) are obtained. It is particularly desirable to use the amino acids at the corresponding positions of CAP derived from various organisms.

Examples of peptides 1 and 2 of the present invention (hereinafter simply referred to as “the peptide of the present invention”) include peptides consisting of all or a part of the amino acid sequence represented by SEQ ID NO: 1 (TQVVWKSTTNIGVGILIVNGTV). The peptide consisting of a part of the amino acid sequence represented by SEQ ID NO: 1 is preferably one containing at least 5 residues from the N-terminal of SEQ ID NO: 1. The number of amino acid residues in the peptide consisting of a part of the amino acid sequence represented by SEQ ID NO: 1 is not particularly limited, and is, for example, 5 to 10, 5 to 11, 5 to 12, 5 to 13, 5 to 14, 5 to 15, 5 to 16, 5 to 17, 5 to 18, 5 to 19, 5 to 20, or 5 to 21.

Examples of the peptide of the present invention also include peptides consisting of an amino acid sequence having deletion, substitution, insertion, and/or addition of 1 to 10, 1 to 5, 1 to 4, 1 to 3, 1 to 2, or 1 amino acid in the amino acid sequence represented by SEQ ID NO: 1, and having at least one of a plant stem cell-inducing effect, a plant environmental stress tolerance-enhancing effect, a plant pest resistance-inducing effect, a germination-promoting effect, a plant transformation efficiency-enhancing effect, and a plant growth-promoting effect. In the case of amino acid substitution, it is considered that the activity of the original polypeptide is likely to be maintained if the amino acid is substituted with an amino acid of similar properties. Techniques of deletion, substitution, insertion, and/or addition of one or two or more amino acids in specific amino acid sequences are known.

The peptide of the present invention also includes salts thereof. Examples of the “salts” include sodium salts, potassium salts, calcium salts, hydrochlorides, sulfates, nitrates, magnesium salts, ammonium salts, phosphates, and organic acid salts (acetate, trifluoroacetate, citrate, maleate, oxalate, malate, lactate, succinate, propionate, fumarate, formate, picrate, benzoate, benzenesulfonate, etc.) of the peptide.

The peptide of the present invention also includes derivatives thereof. The term “derivatives” as used herein refers to those obtained by modifying the functional groups of the peptide of the present invention by modification, addition, mutation, substitution, deletion, or the like by known methods. Examples include those obtained by modifying the N-terminal, C-terminal, or amino acid side chain of the peptide of the present invention with a protecting group or the like. Examples of derivatives include those undergoing acetylation, palmitoylation, amidation, myristylation, dansylation, acrylation, biotinylation, phosphorylation, anilidation, succinylation, benzyloxycarbonylation, formylation, nitration, sulfonation, aldehydration, glycosylation, cyclization, monomethylation, dimethylation, trimethylation, guanidinylation, maleylation, trifluoroacetylation, trinitrophenylation, carbamylation, polyethylene glycolation, acetoacetylation, labeling (e.g., with fluorescent dyes), and the like.

The amino acids that constitute the peptide of the present invention may be L-form or D-form. Further, the amino acids that constitute the polypeptide of the present invention are not limited to natural amino acids, and may be non-natural amino acids.

The peptide of the present invention can be produced by using a known synthesis method such as solid-phase synthesis or liquid-phase synthesis, or by culturing a transformant introduced with a gene encoding the peptide. Examples of the host for producing the transformant include E. coli, yeast, mammalian cells, plant cells, insect cells, and the like.

The produced peptide can be purified by affinity chromatography, ion exchange chromatography, hydroxyapatite column chromatography, ammonium sulfate salting-out, or the like.

The plant stem cell inducer, plant environmental stress tolerance (e.g., drought tolerance, osmotic tolerance, salt tolerance, and freezing tolerance) enhancer, plant pest resistance inducer, germination promoter, plant transformation efficiency enhancer, and plant growth promoter of the present invention (hereinafter also collectively referred to as “the agent of the present invention”) comprise the above peptide.

The method for producing plant stem cells, method for enhancing the environmental stress tolerance (e.g., drought tolerance, osmotic tolerance, salt tolerance, and freezing tolerance) of a plant body, plant pest control method, and method for promoting the growth of a plant body of the present invention each comprise applying the above peptide to the plant body. The method for promoting plant seed germination of the present invention comprises applying the above peptide to plant seeds. The method for transforming a plant body of the present invention comprises transforming the plant body in the presence of the above peptide.

The peptide of the present invention has an excellent plant stem cell-inducing effect, plant environmental stress tolerance (e.g., drought tolerance, osmotic tolerance, salt tolerance, or freezing tolerance)-enhancing effect, plant pest resistance-inducing effect, germination-promoting effect, plant transformation efficiency-enhancing effect, and plant growth-promoting effect. Therefore, the peptide of the present invention can be suitably used as an active ingredient of plant stem cell inducers, plant environmental stress tolerance enhancers, plant pest resistance inducers, germination promoters, plant transformation efficiency enhancers, and plant growth promoters.

Since most species of animals and plants have the CAP gene in common, most plants also have receptors for CAP. Therefore, the peptide of the present invention is expected to be able to, for various plants, induce stem cell transformation, enhance environmental stress tolerance, enhance pest resistance, promote germination, enhance transformation efficiency, and promote growth.

As components other than the peptide of the present invention, the agent of the present invention can suitably contain, if necessary, a support, surfactant, wetting agent, preservative, binder, stabilizer, coloring agent, emulsifier, dispersant, penetrant, thickener, antifoaming agent, and the like. Moreover, the agent of the present invention can be suitably formulated into a wettable powder, granule wettable powder, flowable agent, emulsion, dusting powder, dust formulation, aerosol, paste agent, suspension, solution, or the like by a known method. The agent of the present invention can be used as it is, or used after dilution with a diluent to a predetermined concentration. In addition to the peptide of the present invention, the plant pest resistance inducer of the present invention may further contain a pesticide, such as an insecticide or miticide.

The content of the peptide of the present invention in the plant stem cell inducer of the present invention is not particularly limited as long as the effects of the present invention are obtained, and is preferably 100 to 1000 nM, and more preferably 100 to 600 nM. The content of the peptide of the present invention in the environmental stress tolerance enhancer of the present invention is not particularly limited as long as the effects of the present invention are obtained, and is preferably 1 to 1000 nM, and more preferably 100 to 600 nM. The content of the peptide of the present invention in the plant pest resistance inducer of the present invention is not particularly limited as long as the effects of the present invention are obtained, and is preferably 1 to 600 nM, and more preferably 10 to 500 nM.

The content of the peptide of the present invention in the germination promoter of the present invention is not particularly limited as long as the effects of the present invention are obtained, and is preferably 0.1 to 1,000 μM, and more preferably 10 to 100 μM. The content of the peptide of the present invention in the transformation efficiency enhancer of the present invention is not particularly limited as long as the effects of the present invention are obtained, and is preferably 20 to 10,000 nM, and more preferably 50 to 1,000 nM. The content of the peptide of the present invention in the plant growth promoter of the present invention is not particularly limited as long as the effects of the present invention are obtained, and is preferably 0.1 to 5 μM, and more preferably 0.5 to 1 μM.

The method for applying the peptide of the present invention to a plant body is not particularly limited as long as the effects of the present invention are obtained. Examples include spraying (atomizing) to plant bodies, spraying on the soil surface, injection into the soil, blasting to plant seeds, smearing to plant seeds, immersion of plant seeds, and the like. The peptide of the present invention may be applied to all or a part of the plant body, and a part of the plant body includes leaves, bark, stems, trunks, fruits, seeds, flowers, roots, and the like. The peptide of the present invention may be applied to the plant body only once or repeatedly.

In the method for producing plant stem cells, it is preferable that the plant body is immersed in a solution containing the peptide of the present invention. Further, in the method for enhancing the environmental stress tolerance of a plant body, it is preferable that a solution containing the peptide of the present invention is sprayed (atomized) to the plant body, and that plant seeds are immersed in the solution. On the other hand, in the plant pest control method, it is preferable that a solution containing the peptide of the present invention is sprayed (atomized) to the plant body. In the method for promoting plant seed germination, it is preferable that plant seeds are immersed in a solution containing the peptide of the present invention. In the method for transforming a plant body, it is preferable that transformation is performed using an Agrobacterium solution containing the peptide of the present invention. On the other hand, in the method for promoting the growth of a plant body, it is preferable that a solution containing the peptide of the present invention is directly sprayed (atomized) to the plant body.

In the method for transforming a plant body of the present invention, the method of transformation is not particularly limited. In particular, it is preferable to perform transformation by an Agrobacterium method. The Agrobacterium method can be carried out by using a known means.

The time for immersing a plant body in a solution containing the peptide of the present invention is, for example, about 24 to 96 hours for inducing stem cell transformation, about 0.5 to 16 hours for enhancing drought tolerance, and about 1 to 10 minutes for enhancing osmotic tolerance. Further, the time for promoting plant seed germination is about 5 minutes to 5 hours.

The amounts of the plant stem cell inducer, environmental stress tolerance enhancer, germination promoter, transformation efficiency enhancer, and plant growth promoter of the present invention applied can be suitably selected depending on the target plant, dosage form, application method, etc. Further, the amount of the plant pest resistance inducer of the present invention applied can be suitably selected depending on the target pest, target pathogen, target harmful insect, target plant, dosage form, degree of pest occurrence, application method, etc.

Plants targeted by the agent of the present specification are not particularly limited, and may be angiosperms or gymnosperms. Angiosperms include both dicotyledonous and monocotyledonous plants. Examples of the targeted plants include the following: vegetables, such as leafy greens (e.g., Chinese cabbage, spinach, cabbage, lettuce, komatsuna, garland chrysanthemum, mizuna, welsh onion, onion, broccoli, and asparagus), root vegetables (e.g., carrot, radish, burdock, eddo, yam, lotus root, and turnip), and fruit vegetables (e.g., tomato, cherry tomato, eggplant, pepper, cucumber, watermelon, strawberry, pumpkin, melon, and sweet corn); fruit trees, such as apples, pears, grapes, and persimmons; legumes, such as soybeans, green beans, adzuki beans, fava beans, peanuts, and kidney beans; tea; tobacco; cotton; cereal grasses, such as barley, wheat, oat, and rye; flowering plants, such as pansies, marigolds, salvias, petunias, periwinkles, chrysanthemums, carnations, roses, gentians, baby's breath, gerberas, statice, prairie gentian, alstroemeria, lilies, tulips, cyclamens, gladiolus, freesias, primulas, dendrobiums, begonias, cymbidium, poinsettias, phalaenopsis, violas, daisies, scoparia, and calibrachoa; succulent plants, such as cacti; lawn grasses, such as cool-season grass and Korean lawn grass; and afforestation plants. Thus, the agent of the present invention can be widely used for agricultural crops, cultivars, research plants, and the like.

Plant diseases targeted by the plant pest resistance inducer of the present invention are not particularly limited as long as the effects of the present invention are obtained. Examples include the following plant diseases:
blast (Pyricularia oryzae), sheath blight (Thanatephorus cucumeris), brown spot (Cochliobolus miyabeanus), bakanae disease (Gibberella fujikuroi), seedling blight (Pythium spp., Fusarium spp., Trichoderma spp., Rhizopus spp., Rhizoctonia solani, etc.), false smut (Claviceps virens), and kernel smut (Tilletia barclayana) of rice;
powdery mildew (Erysiphe graminis f. sp. hordei), rust (Puccinia striiformis, Puccinia graminis, Puccinia recondita, Puccinia hordei), leaf stripe (Pyrenophora graminea), net blotch (Pyrenophora teres), fusarium head blight (Fusarium graminearum, Fusarium culmorum, Fusarium avenaceum, Microdochium nivale), typhula snow blight (Typhula incarnata, Typhula ishikariensis, Micronectriella nivalis), loose smut (Ustilago nuda, Ustilago tritici, Ustilago nigra, Ustilago avenae), bunt (Tilletia caries, Tilletia pancicii), eye spot (Pseudocercosporella herpotrichoides), foot-rot (Rhizoctonia cerealis), scald (Rhynchosporium secalis), leaf blotch (Septoria tritici), glume blotch (Leptosphaeria nodorum), seedling blight (Fusarium spp., Pythium spp., Rhizoctonia spp., Septoria nodorum, Pyrenophora spp.), take-all (Gaeumannomyces graminis), anthracnose (Colletotrichum graminicola), ergot (Claviceps purpurea), and spot blotch (Cochliobolus sativus) of wheat;
fusarium head blight (Fusarium graminearum, etc.), seedling blight (Fusarium avenaceum, Penicillium spp., Pythium spp., Rhizoctonia spp.), rust (Puccinia sorghi), leaf blight (Cochliobolus heterostrophus), smut (Ustilago maydis), anthracnose (Colletotrichum graminicola), and northern leaf spot (Cochliobolus carbonum) of corn;
gray mold (Botrytis cinerea) of tomato, cucumber, legumes, strawberry, potato, cabbage, eggplant, lettuce, etc.;
stem rot (Sclerotinia sclerotiorum) of tomato, cucumber, legumes, strawberry, potato, rapeseed, cabbage, eggplant, lettuce, etc.;
damping-off (Rhizoctonia spp., Pythium spp., Fusarium spp., Phytophthora spp., Sclerotinia sclerotiorum, etc.) of various vegetables, such as tomato, cucumber, legumes, radish, watermelon, eggplant, rapeseed, green pepper, spinach, and sugar beet;
purple stain (Cercospora kikuchii), sphaceloma scab (Elsinoe glycines), pod and stem blight (Diaporthe phaseolorum), Rhizoctonia root rot (Rhizoctonia solani), root rot (Phytophthora megasperma), downy mildew (Peronospora manshurica), rust (Phakopsora pachyrhizi), and anthracnose (Colletotrichum truncatum) of soybean;
downy mildew (Pseudoperonospora cubensis), powdery mildew (Sphaerotheca fuliginea), anthracnose (Colletotrichum lagenarium), gummy stem blight (Mycosphaerella melonis), fusarium wilt (Fusarium oxysporum), and phytophthora blight (Phytophthora parasitica, Phytophthora melonis, Phytophthora nicotianae, Phytophthora drechsleri, Phytophthora capsici, etc.) of gourds;
leaf spot (Alternaria brassicae) of rapeseed;
leaf spot (Alternaria brassicae, etc.), white spot (Cercosporella brassicae), blackleg (Leptosphaeria maculans), clubroot (Plasmodiophora brassicae), and downy mildew (Peronospora brassicae) of brassicaceae vegetables;
bacterial leaf spot (Pseudomonas syringae pv tomato DC3000), early blight (Alternaria solani), leaf mold (Cladosporium fulvum), late blight (Phytophthora infestans), fusarium wilt (Fusarium oxysporum), root rot (Pythium myriotylum, Pythium dissotocum), and anthracnose (Colletotrichum phomoides) of tomato;
damping-off (Rhizoctonia solani) and yellows (Fusarium oxysporum) of cabbage;
bottom rot (Rhizoctonia solani) and yellows (Verticillium dahliae) of Chinese cabbage;
rust (Puccinia allii), purple blotch (Alternaria porri), southern blight (Sclerotium rolfsii), and white tip disease (Phytophthora porri) of welsh onion;
powdery mildew (Sphaerotheca fuliginea, etc.), leaf mold (Mycovellosiella nattrassii), late blight (Phytophthora infestans), and brown rot (Phytophthora capsici) of eggplant;
leaf spot (Mycosphaerella personatum) and brown leaf spot (Cercospora arachidicola) of peanut;
powdery mildew (Erysiphe pisi), downy mildew (Peronospora pisi), and mycosphaerella blight (Mycosphaerella pinodes) of pea;
downy mildew (Peronospora viciae) and phytophthora rot (Phytophthora nicotianae) of broad bean;
anthracnose (Colletotrichum lindemuthianum) of kidney bean;
early blight (Alternaria solani), black scurf (Rhizoctonia solani), late blight (Phytophthora infestans), silver scurf (Spondylocladium atrovirens), dry rot (Fusarium oxysporum, Fusarium solani), and powdery scab (Spongospora subterranea) of potato;
powdery mildew (Sphaerotheca humuli), phytophthora rot (Phytophthora nicotianae), anthracnose (Glomerella cingulata), and fruit rot (Pythium ultimum Trow var. ultimum) of strawberry;
downy mildew (Plasmopara viticola), rust (Phakopsora ampelopsidis), powdery mildew (Uncinula necator), anthracnose (Elsinoe ampelina), ripe rot (Glomerella cingulata), black rot (Guignardia bidwellii), dead arm (Phomopsis viticola), flyspeck (Zygophiala jamaicensis), gray mold (Botrytis cinerea), bud blight (Diaporthe medusaea), violet root rot (Helicobasidium mompa), and white root rot (Rosellinia necatrix) of grape;
powdery mildew (Podosphaera leucotricha), scab (Venturia inaequalis), alternaria blotch (Alternaria alternata (apple pathotype)), rust (Gymnosporangium yamadae), blossom blight (Monilinia mali), canker (Valsa ceratosperma), ring rot (Botryosphaeria berengeriana), bitter rot (Colletotrichum acutatum), flyspeck (Zygophiala jamaicensis), sooty blotch (Gloeodes pomigena), fruit spot (Mycosphaerella pomi), violet root rot (Helicobasidium mompa), white root rot (Rosellinia necatrix), canker (Phomopsis mali, Diaporthe tanakae), and blotch (Diplocarpon mali) of apple;
anthracnose (Gloeosporium kaki), leaf spot (Cercospora kaki, Mycosphaerella nawae), and powdery mildew (Phyllactinia kakikora) of persimmon;
purple blotch (Alternaria alternata (Japanese pear pathotype)), scab (Venturia nashicola), rust (Gymnosporangium haraeanum), physalospora canker (Physalospora piricola), canker (Diaporthe medusaea, Diaporthe eres), and crown rot (Phytophthora cactorum) of pear;
melanose (Diaporthe citri), common green mold (Penicillium digitatum), blue mold (Penicillium italicum), and scab (Elsinoe fawcettii) of citrus;
stem rot (Sclerotinia sclerotiorum) of sunflower;
scab (Cladosporium carpophilum), phomopsis rot (Phomopsis sp.), phytophthora rot (Phytophthora sp.), and anthracnose (Gloeosporium laeticolor) of peach;
anthracnose (Glomerella cingulata), young-fruit rot (Monilinia kusanoi), and brown rot (Monilinia fructicola) of cherry;
black spot (Diplocarpon rosae), powdery mildew (Sphaerotheca pannosa, Colletotrichum gloesporioides), downy mildew (Peronospora sparsa), and phytophthora rot (Phytophthora megasperma) of rose;
brown spot (Septoria chrysanthemi-indici), rust (Puccinia horiana), and crown rot (Phytophthora cactorum) of chrysanthemum; and
sooty mold of Quercus myrsinaefolia.

Plant pests targeted by the plant pest resistance inducer of the present invention are not particularly limited as long as the effects of the present invention are obtained. The plant pest resistance inducer of the present invention can be widely used for known pests, and is particularly suitable for agricultural pests.

Since the peptide of the present invention has an excellent plant stem cell-inducing effect, plant environmental stress tolerance-enhancing effect, and plant pest resistance-inducing effect, exposing plants to the peptide of the present invention can transform differentiated cells of the plants into stem cells, enhance the environmental stress tolerance of the plants, and enhance the pest resistance of the plants. Further, the peptide of the present invention has an excellent germination-promoting effect, plant transformation efficiency-enhancing effect, and plant growth-promoting effect, exposing plants to the peptide of the present invention can promote the germination of plant seeds, enhance plant transformation efficiency, and promote the growth of plant bodies. Therefore, it is an extremely simple method, and the peptide of the present invention has about 6 to 25 amino acid residues and is thus easy to synthesize.

The peptide of the present invention does not require the process of transformation, and thus can basically be applied to any plants, and can be used both inside and outside the laboratory.

Stem cell transformation requires only short-term exposure of plants to the peptide of the present invention, and is extremely simple and fast-acting. Further, since all cells infiltrated with the peptide of the present invention are transformed into stem cells, it is easy to identify cells that have differentiated into stem cells. Because it is possible to transform differentiated cells of various plants, including plants that are difficult to initialize and plants that are difficult to transform, into stem cells, the efficiency of tissue culture is considered to increase. Breed improvement in the state of callus becomes possible by performing stem cell transformation.

Moreover, the peptide of the present invention can enhance plant environmental stress tolerance, and makes it possible to protect crops from frost damage and cultivate crops in arid regions.

The peptide of the present invention can enhance the pest resistance of plants, and can protect crops from pests. Further, the peptide of the present invention is not toxic to living organisms unlike pesticides, and is decomposed by microorganisms in the environment, which causes no problem with residue. The peptide of the present invention can be applied to not only agricultural production sites, but also food crops, because it is not genetically modified and is safe.

The peptide of the present invention can promote the germination of plant seeds. It is possible not only to germinate old seeds, but also to revive plants thought to be extinct.

The peptide of the present invention can enhance plant transformation efficiency, allows transformation of plants that have not been able to transform, can develop research on plants that have not been able to be studied due to their inability to transform, and makes difficult-to-transform plants useful by genome editing or the like. In addition, the peptide of the present invention can promote the growth of plants, which makes it possible to economically increase the production of crops without excessive fertilization.

### EXAMPLES

Examples are provided below to describe the present invention in more detail. However, the present invention is not limited to these Examples etc.

### Test Example 1

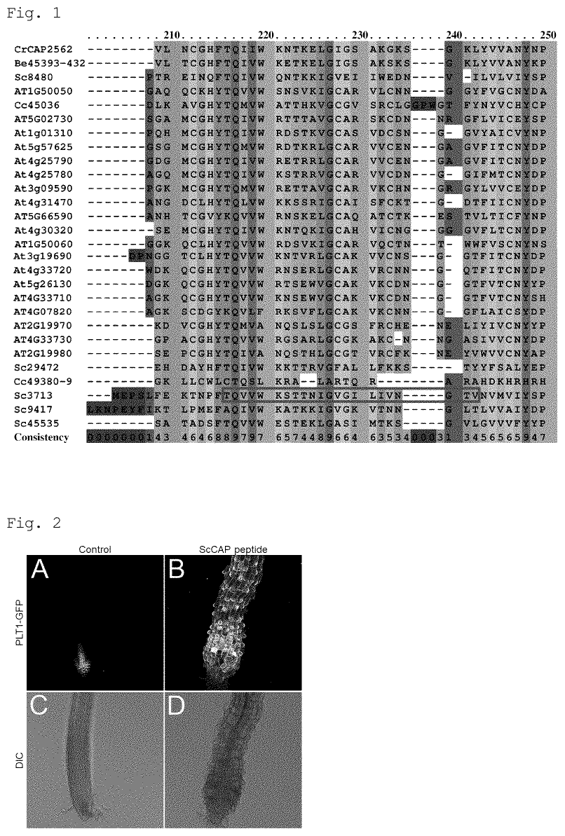

The CAP protein sequences of Arabidopsis thaliana, Schlechtendalia chinensis, Borboryctis euryae, Caloptilia cecidophora, and Caloptilia ryukyuensis were aligned. As a result, FIG. 1 shows the conserved region conserved between the CAP proteins. The synthetic peptide in the boxed area in FIG. 1 (hereinafter referred to as “CAP peptide”) was used in the following experiments.

Arabidopsis thaliana roots were treated with CAP peptide (TQVVWKSTTNIGVGILIVNGTV). Arabidopsis thaliana expressing stem cell marker PLT1-GFP was immersed in water or a 600 nM peptide aqueous solution for 2 days, and GFP fluorescent images and bright field images were obtained with a confocal laser microscope.

The results are shown in FIG. 2. FIG. 2 indicates that the synthetic peptide from the conserved region of insect CAP induced a gall-like structure, and that differentiated cells were transformed into stem cells.

### Test Example 2

RNA of Arabidopsis thaliana immersed in water or a 600 nM CAP peptide solution for 4 hours was extracted, and gene expression was compared by RNA-seq analysis. Network analysis was then performed on genes with increased expression.

The results showed that immersing the plant in 600 nM CAP peptide for 4 hours increased the expression of genes related to freezing tolerance and drought tolerance (by a factor of 2 or more).

Next, wild-type Arabidopsis thaliana seeds were immersed in water (Control) or 600 nM CAP peptide for 5 minutes and then grown on an excess mannitol-containing medium. Further, wild-type Arabidopsis thaliana seeds were immersed in water (Control) or 600 nM CAP peptide overnight, and then grown on an excess sorbitol-containing medium.

The results are shown in FIG. 3. In the excess mannitol-containing medium (high-osmotic pressure conditions), the plant roots treated with CAP peptide extended more (upper figure in FIG. 3), and in the excess sorbitol-containing medium (dry conditions), the plant roots treated with CAP peptide also extended more (lower figure in FIG. 3).

### Test Example 3

RNA of Arabidopsis thaliana immersed in water or a 600 nM CAP peptide solution for 48 hours was extracted, and gene expression was compared by RNA-seq analysis. Network analysis was then performed.

The results showed that immersing the plant in 600 nM peptide for 48 hours increased the expression of genes related to disease resistance (by a factor of 2 to 3 or more).

Next, Arabidopsis thaliana leaves were sprayed with water/0.02% Silwet L-77 (Control) or 600 nM CAP peptide/0.02% Silwet L-77, and 24 hours later, infected with Pseudomonas syringae pv tomato DC3000 (DC3000) with an OD600 of 0.2×10⁻³. 2 days later, DC3000 obtained from leaf fragments cut into 4-mm-diameter circles was diluted and spotted on LB medium containing kanamycin. After incubation at 28° C. for 24 hours, the number of bacteria per unit area was counted.

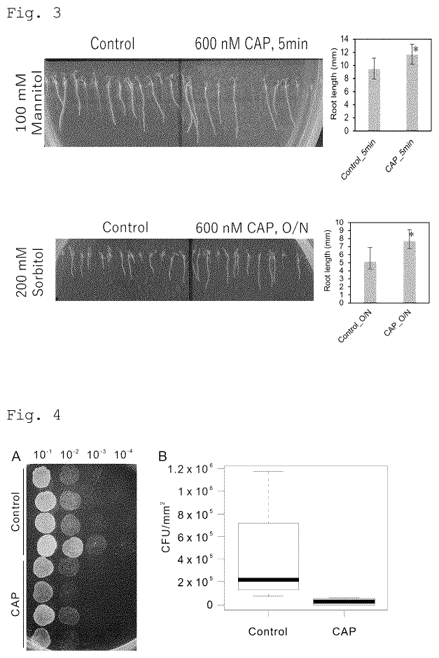

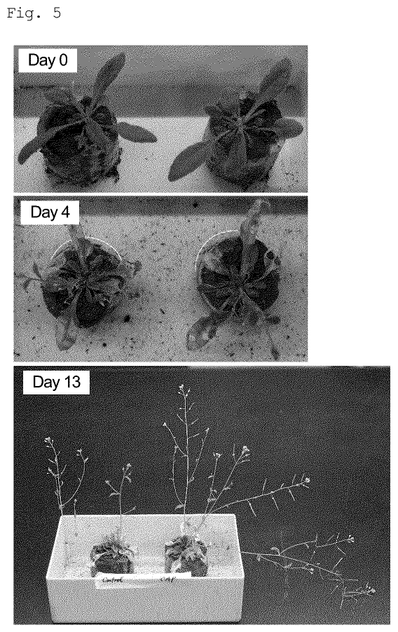

The results are shown in FIG. 4. Spraying Arabidopsis thaliana leaves with a 600 nM CAP peptide solution in advance suppressed the growth of bacteria after infection with plant pathogen Pseudomonas syringae pv tomato DC3000.

### Test Example 4

Arabidopsis thaliana leaves applied with water or 60 nM CAP peptide were inoculated with Pseudomonas syringae DC3000.

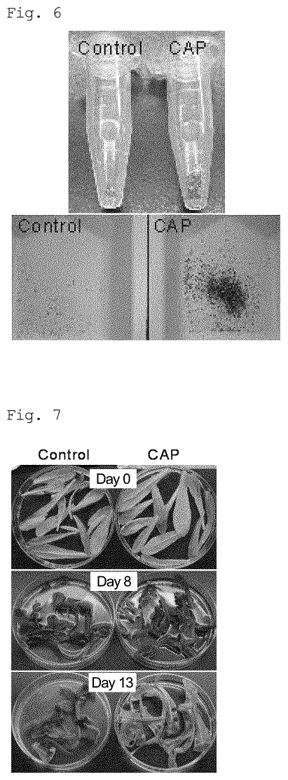

The results are shown in FIGS. 5 and 6. 4 days later, the growth of the pathogen was significantly inhibited in the CAP peptide-treated plant body (middle figure in FIG. 5). At 13 days after infection, the CAP peptide-treated plant body grew much larger than the water treatment (Control) (lower figure in FIG. 5). At 13 days after pathogen infection, the CAP peptide-treated plant body grew much larger and yielded more seeds than the water treatment (Control) (FIG. 6).

### Test Example 5

Brassica rapa leaves were sprayed with water or a 60 nM CAP peptide solution in advance, and one day later, inoculated with plant pathogen Pseudomonas syringae pv tomato DC3000 (DC3000). 2 days after infection, DC3000 obtained from leaf fragments cut into 4-mm-diameter circles was diluted, spotted on LB medium containing kanamycin, and incubated at 28° C. for 24 hours.

The results are shown in FIGS. 7 and 8. When the CAP peptide-treated Brassica rapa was treated with Pseudomonas syringae DC3000, the freshness was maintained for a longer period of time than the water treatment (Control) (FIG. 7). Further, 2 days after pathogen inoculation, the CAP peptide-treated Brassica rapa inhibited bacterial growth 1000 times or more, compared with the water treatment (Control) (FIG. 8).

### Test Example 6

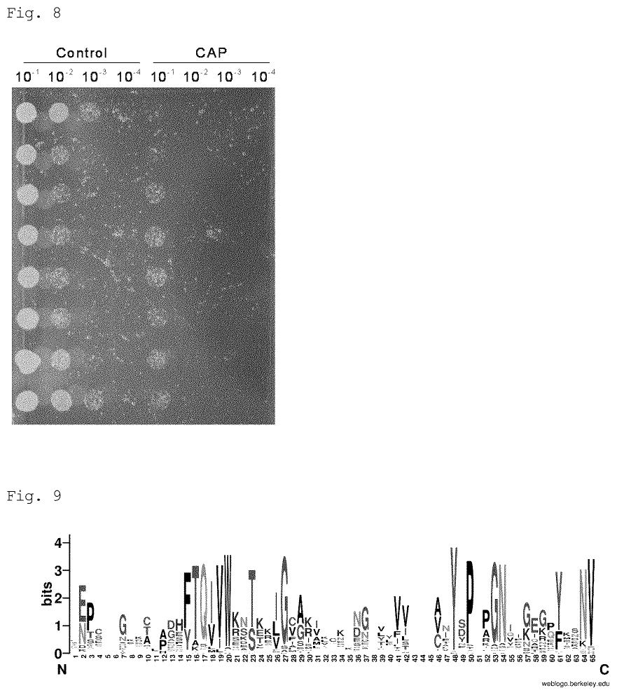

Multiple types (76 types) of CAP proteins derived from Schlechtendalia chinensis, Caloptilia cecidophora, Borboryctis euryae, Caloptilia ryukyuensis, Rhus javanica, and Arabidopsis thaliana were aligned. From the consensus sequence (FIG. 9), FTQIVW (SEQ ID NO: 21) was determined (hereinafter referred to as “CAP6 peptide”). Then, Arabidopsis thaliana leaves were sprayed with 0.02% Silwet L-77 (Control), 60 nM CAP peptide/0.02% Silwet L-77, or 60 nM CAP6 peptide/0.02% Silwet L-77, and 48 hours later, infected with Pseudomonas syringae pv tomato DC3000 (DC3000) with an OD600 of 0.2×10⁻³. One day later, DC3000 obtained from leaf fragments cut into 5-mm-diameter circles was diluted and spotted on LB medium containing kanamycin. 24 hours later, the number of bacteria per unit area was counted.

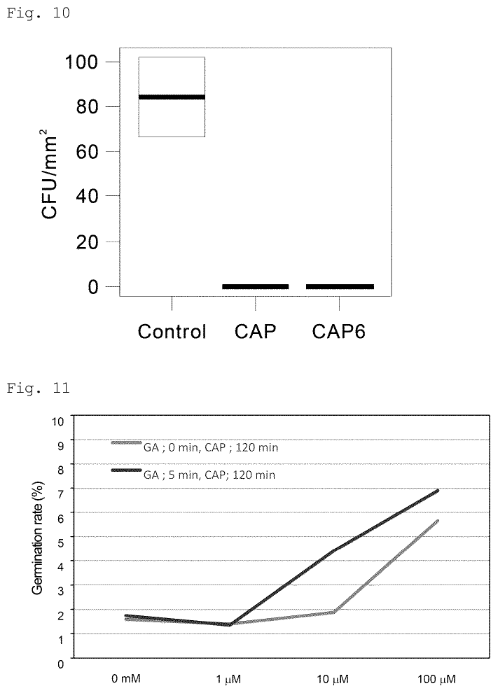

The results are shown in FIG. 10. When a disease tolerance test was performed using CAP6 peptide, a remarkable effect was obtained. Therefore, the peptide sequence showing the effect of the present invention is presumed to be F/Y-T-Q-I/V-V/I-W.

### Test Example 7

Arabidopsis thaliana seeds harvested 10 years before and not naturally germinated were immersed in 0, 1, 10, or 100 μM CAP peptide for 2 hours and then sown. The germination rate was measured 2 weeks later.

The results are shown in FIG. 11. As a result, CAP peptide treatment of the Arabidopsis thaliana seeds resulted in a much higher germination rate than gibberellin treatment (FIG. 11).

### Test Example 8

Agrobacterium introduced with a plasmid expressing GFP-TUB6 protein was mixed with CAP peptide to final concentrations of 50 nM, 1 μM, and 10 μM, and transformed into Rorippa aquatica.

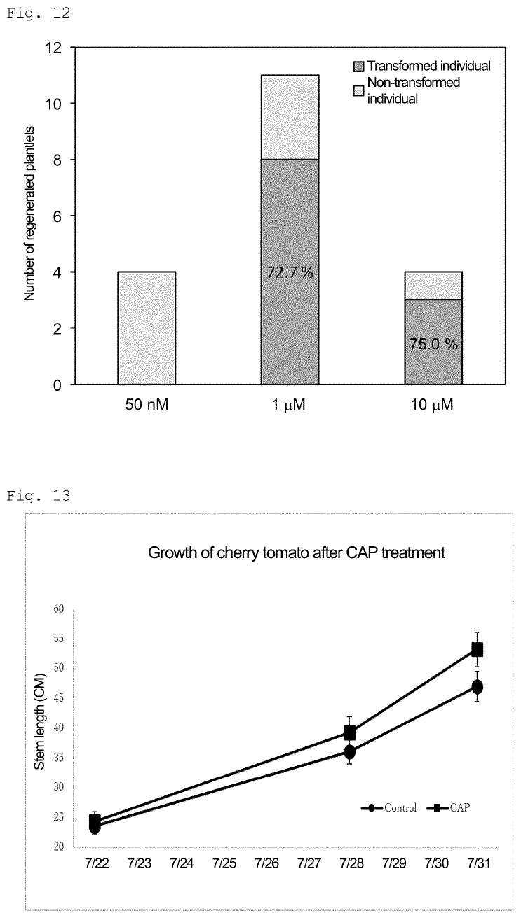

The results are shown in FIG. 12. As a result, the addition of 1 μM CAP peptide resulted in high efficiency transformation (FIG. 12).

### Test Example 9

Cherry tomato Sun Cherry seedlings were purchased, sprayed with 1 μM CAP peptide on Jul. 21, 2021, and then grown in planters in an alley. The second 1 μM CAP peptide treatment was performed on July 28.

The results are shown in FIG. 13. As a result, compared with the CAP-untreated group, the effect of promoting stem extension was significantly confirmed (t-test: p<0.05) in the CAP-treated group only on July 3 after the 1 μM CAP peptide treatment was performed twice (FIG. 13).

### SEQUENCE LISTING

1. A peptide that is a partial peptide of cysteine-rich secretory protein, antigen 5, and pathogenesis-related 1 (CAP), consisting of an amino acid sequence of the conserved region of CAP, the peptide having at least one effect selected from the following effects: a plant stem cell-inducing effect, a plant environmental stress tolerance-enhancing effect, a plant pest resistance-inducing effect, a germination-promoting effect, a plant transformation efficiency-enhancing effect, and a plant growth-promoting effect.

2. The peptide according to claim 1, wherein CAP is secreted from a gall-inducing insect.

3. A peptide comprising an amino acid sequence represented by formula (I):
(X¹)ₙTQX²X³W (I)
wherein X¹ represents phenylalanine or tyrosine, X² represents isoleucine or valine, X³ represents isoleucine or valine, and n represents 0 or 1;
and having 25 or less amino acid residues.

4. The peptide according to claim 1, which is a peptide consisting of all or a part of an amino acid sequence represented by SEQ ID NO: 1.

5. A plant stem cell inducer comprising the peptide according to claim 1.

6. A plant environmental stress tolerance enhancer comprising the peptide according to claim 1.

7. A plant pest resistance inducer comprising the peptide according to claim 1.

8. A germination promoter comprising the peptide according to claim 1.

9. A plant transformation efficiency enhancer comprising the peptide according to claim 1.

10. A plant growth promoter comprising the peptide according to claim 1.

11. A method for producing plant stem cells, comprising applying the peptide according to claim 1 to a plant body.

12. A method for enhancing the environmental stress tolerance of a plant body, comprising applying the peptide according to claim 1 to the plant body.

13. A plant pest control method comprising applying the peptide according to claim 1 to a plant body.

14. A method for promoting plant seed germination, comprising applying the peptide according to claim 1 to plant seeds.

15. A method for transforming a plant body, comprising transforming the plant body in the presence of the peptide according to claim 1.

16. A method for promoting the growth of a plant body, comprising applying the peptide according to claim 1 to the plant body.


SEQUENCE LISTING (21 sequences)

SEQ ID NO: 1 - protein, 22 aa, Artificial Sequence
  partial peptide of CAP
TQVVWKSTTNIGVGILIVNGTV

SEQ ID NO: 2 - protein, 166 aa, Arabidopsis thaliana
MKMFKSPQTLILSVIVLFLAFAVPLKAQDQPQDYLDEHNRARTQVGVPPMKWHAGAEQYAWNYAQQRKGDCSLTHSNSNGLYGENLAWSGGALSGAEAVKLWVNEKSDYIYASNTCSDGKQCGHYTQVVWRTSEWVGCAKVKCDNGGTFVTCNYYPPGNYRGRWPY

SEQ ID NO: 3 - protein, 226 aa, Arabidopsis thaliana
MNTFKTPFLIIVAISFLVLATNAQNAQQDYLNTHNTARAQVGVANVVWDTVVAAYATNYANARKVDCSLTPSTGGSYGENLANGNNALFTGVAAVNLWVNEKPYYNYTANACIGAQQCKHYTQVVWSNSVKIGCARVLCNNGGYFVGCNYDASAALKSRKITASVRQTRGLGASAACGQLRNKFQKSPSLATAEGEVIEARLRSEDFDNSCIGFAFTYICDACVGL

SEQ ID NO: 4 - protein, 205 aa, Arabidopsis thaliana
MSSSSLYHPFCLSISSVLLLLLLIFSGEFPSTAGTSSPDTKAAAARATNRGRRNKQSAEFLLAHNAARVASGASNLRWDQGLARFASKWAKQRKSDCKMTHSGGPYGENIFRYQRSENWSPRRVVDKWMDESLNYDRVANTCKSGAMCGHYTQIVWRTTTAVGCARSKCDNNRGFLVICEYSPSGNYEGESPFDIPKITLKLDKI

SEQ ID NO: 5 - protein, 241 aa, Arabidopsis thaliana
MELRKRKIRAPLHIFVGITFFLLQLWSGTAQINHEDSSTKPSVKNSPSATSTRLLLSPPSFTGNRFSFRFRWRRIRRRNRVNRASREFLIAHNLVRARVGEPPFQWDGRLAAYARTWANQRVGDCRLVHSNGPYGENIFWAGKNNWSPRDIVNVWADEDKFYDVKGNTCEPQHMCGHYTQIVWRDSTKVGCASVDCSNGGVYAICVYNPPGNYEGENPFGSYDDQIGLARDDPPAVIGGMA

SEQ ID NO: 6 - protein, 207 aa, Arabidopsis thaliana
MATTSSTKQVIVFTISLLLVAFQAVHADYYRQRPPVTPTPYVPKPRYPLPSPSPKPVYRPPTTPSLPAGSIARLFLDPHNALRSGLGLPPLIWDGKLASYATWWANQRRYDCSLTHSTGPYGENLFWGSGSSWAPGFAVQSWIVEGRSYNHNTNSCDGSGMCGHYTQMVWRDTKRLGCARVVCENGAGVFITCNYDPPGNYVGEKPY

SEQ ID NO: 7 - protein, 210 aa, Arabidopsis thaliana
MATTTSTQAIVFAIALFLVAIQAVHADYYRHRPQRPITPSPPPYVAKPQPLPTPSPKPILYQPPPTYQPPTGSFEQQFLDPHNTVRGGLGLPPLVWDVKIASYATWWANQRRYDCSLTHSTGPYGENLFWGSGSDFTSTFAVESWTVEAKSYNHMTNTCEGDGMCGHYTQIVWRETRRLGCARVVCENGAGVFITCNYDPPGNYVGEKPY

SEQ ID NO: 8 - protein, 190 aa, Arabidopsis thaliana
MSSSYLRVILLLGALNVVVSLSITNSLITKSATLGQVFRICKNLCPGCDHDSLQFLFRHNLVRAARFEPPLIWDRRLQNYAQGWANQRRGDCALRHSVSNGEFNLGENIYWGYGANWSPADAVVAWASEKRFYHYGSNTCDAGQMCGHYTQIVWKSTRRVGCARVVCDNGGIFMTCNYDPPGNYIGQKPY

SEQ ID NO: 9 - protein, 186 aa, Arabidopsis thaliana
MSVTLSHPSSCLLLLFLLFSGHPSVLGTSIPDAVNTAARLVNRARRAKLSREFLQAHNDARVSSGVPTLGWDRDLARFADKWAKQRKSDCSMIHSGGPYGENIFWHRRKKTWSPEKVVTRWFEERFNYDVKTNTCAPGKMCGHYTQMVWRETTAVGCARVKCHNGRGYLVVCEYDPRGNYEGERPF

SEQ ID NO: 10 - protein, 185 aa, Arabidopsis thaliana
MEQKTLIFIIIIIVISTPLPSLSFQIPSNRTPTTSTLIFSQDKALARNTIQQQFLRPHNILRAKLRLPPLKWSNSLALYASRWARTRRGDCKLIHSGGPYGENLFWGSGKGWTPRDAVAAWASEMKYYDRRTSHCKANGDCLHYTQLVWKKSSRIGCAISFCKTGDTFIICNYDPPGNIVGQPPF

SEQ ID NO: 11 - protein, 185 aa, Arabidopsis thaliana
MALTHHIIFVALLVISVKAISPAAKLKPKQIVSTSPPPPPTISAAAKAFTDAHNKARAMVGVPPLVWSQTLEAAASRLARYQRNQKKCEFASLNPGKYGANQLWAKGLVAVTPSLAVETWVKEKPFYNYKSDTCAANHTCGVYKQVVWRNSKELGCAQATCTKESTVLTICFYNPPGNVIGQKPY

SEQ ID NO: 12 - protein, 161 aa, Arabidopsis thaliana
MAFPSCVSVAITAMMLLVTCCHCATYQEQFMGPQNAARAHLRLKPLKWDAKLARYAQWWANQRRGDCALTHSNGPYGENLFWGSGNRWGPSQAAYGWLSEARSYNYRSNSCNSEMCGHYTQIVWKNTQKIGCAHVICNGGGGVFLTCNYDPPGNFLGRKPY

SEQ ID NO: 13 - protein, 161 aa, Arabidopsis thaliana
MNTFKTPFLVIVAISFLVVATNAQNTPQDYLNSHNTARAQVGVPNVVWDTTLAAYALNYSNFRKADCNLVHSNGPYGENLAKGSSSSFSAISAVKLWVDEKPYYSYAYNNCTGGKQCLHYTQVVWRDSVKIGCARVQCTNTWWFVSCNYNSPGNWVGEYPY

SEQ ID NO: 14 - protein, 161 aa, Arabidopsis thaliana
MSLLKTNILFLLAIALFYGSLAEDLQQQFLEAHNEARNEVGLDPLVWDDEVAAYAASYANQRINDCALVHSNGPFGENIAMSSGEMSAEDAAEMWINEKQYYDYDSNTCNDPNGGTCLHYTQVVWKNTVRLGCAKVVCNSGGTFITCNYDPPGNYIGEKPF

SEQ ID NO: 15 - protein, 163 aa, Arabidopsis thaliana
MKIFNSSQNLFLAITFFLVLIVHLKAQDSPQDFLAVHNRARAEVGVGPLRWDEKVAAYARNYANQRKGDCAMKHSSGSYGENIAWSSGSMTGVAAVDMWVDEQFDYDYDSNTCAWDKQCGHYTQVVWRNSERLGCAKVRCNNGQTFITCNYDPPGNWVGEWPY

SEQ ID NO: 16 - protein, 166 aa, Arabidopsis thaliana
MKMFISPQTLVLLALALVLAFAVPLKAQDRRQDYLDVHNHARDDVSVPHIKWHAGAARYAWNYAQRRKRDCRLIHSNSRGRYGENLAWSSGDMSGAAAVRLWVREKSDYFHKSNTCRAGKQCGHYTQVVWKNSEWVGCAKVKCDNGGTFVTCNYSHPGNVRGRRPY

SEQ ID NO: 17 - protein, 160 aa, Arabidopsis thaliana
MFNSYPNLLVLAIALALSFSVPLKAQDQPQDYFNAHNRARVSVGVSPLMWSQTLTAYAQAYAEKRRDCGLFLSGGPYGETIKADIIDFSAEEFVSTFLNQKSDYDYTTNTCRAGKSCDGYKQVLFRKSVFLGCAKVKCNNGGFLAICSYDPSVILSERPF

SEQ ID NO: 18 - protein, 177 aa, Arabidopsis thaliana
MSSFRYTFVLLTLLSVLLTRAYGLPRVRPIKDVQPRKTLKVHNQIRAAVGVAPLKWNKTVAAYAQKFANRQAKAGVCDYSSMRHSDGPYGENIAAGWVQPKDQMSGPIAAKYWLTEKPNYDHATNKCKDVCGHYTQMVANQSLSLGCGSFRCHENELIYIVCNYYPMPVGDENTRPY

SEQ ID NO: 19 - protein, 172 aa, Arabidopsis thaliana
MFHKVAIETLVLLLLINYLTQIDVSSAQYSQYPQSHEYPDSYLRPHNAARAAVKVKPLRWDFGIATVAQDYANHLASGPCSLEHSSGPYGENLAFGSGDMSAAQAVAMWVHEKSYYDFYSNSCHGPACGHYTQVVWRGSARLGCGKAKCNNGASIVVCNYDPAGNYIGARPY

SEQ ID NO: 20 - protein, 165 aa, Arabidopsis thaliana
MSFSGYSFIVLTLLSIVLTQIYGLRSFSRMDDLQPAETLAVHNQIRAADQKLAAHAQRYANVRSQDCAMKYSTDGTYGENIAAGWVQPMDTMSGPIATKFWFTEKPYYNYATNKCSEPCGHYTQIVANQSTHLGCGTVRCFKNEYVWVVCNYAPRPMGDANTRPY

SEQ ID NO: 21 - protein, 6 aa, Artificial Sequence
  consensus sequence of CAP
FTQIVW